(19) United States
(12) Patent Application Publication
(10) Pub. No.: US 2010/0183691 A1
(43) Pub. Date: Jul. 22, 2010

(54) Use of Titanium metal fine-particles for increasing the effect of Germicidal medicines used for human skin dermatosis, skin infection and traumatism

(76) Inventor: Xiaosong Zhu, Mississauga (CA)

Correspondence Address: GOWAN INTELLECTUAL PROPERTY, 1075 NORTH SERVICE ROAD WEST, SUITE 203, OAKVILLE, ON, L6M-2G2, CA

(21) Appl. No.: 12/683,478

(22) Filed: Jan. 7, 2010

(30) Foreign Application Priority Data
Jan. 22, 2009 (CN) 200910045915.3

(51) Int. Cl. A61K 9/00; A61K 9/70; A61K 33/24; B32B 5/00; A61P 31/10; A61P 31/00; A61P 17/00; C22C 14/00

(52) U.S. Cl. 424/404; 424/443; 424/617; 424/447; 428/402; 420/417

(57) ABSTRACT

The use of Titanium metal fine-particles for increasing of effect of the germicidal medicines used for human skin, skin infection and traumatism, is provided. The application of the Titanium metal fine-particles layer being laid on or close to skin, in combination with a medicine, functions to restrain bacteria breeding and multiplying, accelerate skin and dermic-organism metabolism, and promoting capillary purification. By these functions, an environment which is harmful and fatal for bacteria breeding/multiplying will be developed on the human body skin and dermic-organism. These functions are demonstrated by the increased effects of the germicidal medicines which are used for human skin dermatosis, skin infection and traumatism.

### FIELD OF THE INVENTION

This invention relates to the use of fine particles of titanium metal for increasing the effect of germicidal medicines used for human skin dermatosis, skin infection and traumatism.

### BACKGROUND OF THE INVENTION

In the marketplace, there are many kinds of antifungal and/or antibiotics medicines used for dermatosis, inflammation, skin infection, skin wound and traumatism, and the like, which are available in many different styles such as, for example, in liquids, cream or powder, etc. To date, even though these medicines have shown beneficial effects, not all properties of these products are satisfactory to the patient and/or doctor due to many limits in their application, such as length of application times, consistency, medicine style, patient's body condition, and the like. In addition, some medicines need to be continuously applied for longer times which can lead to other problems such as unfavourable patient daily life, medicine-resistance, etc. As such, both patients and doctors continuously seek to find ways to increase the effect of a medicine without increasing in dosage.

In another technological area, Phild Co. Ltd. Of Japan has developed patent technologies for producing Titanium metal micro-dispersions of small fine powders of Titanium metal in water (see for example, Japanese Patent Application Laid-open No. 2001-314878 and No. 2005-232183; International PCT patent Publication. Nos. WO03/033417, WO20/078884, and WO02/078883; and U.S. Pat. No. 7,300,491 B2).

Phild Co. Ltd. also has developed so-called “healthy fiber products” by applying the Titanium metal micro-dispersions of water and small fine powder of Titanium metal, to a fibrous substrate (Japanese Patent Application Laid-open No. 2002-020969, No. 2005-278843, and No. 2005-287901; International PCT Pub. No. WO02/078481; Registered Japanese Utility Model No. 3097668, No. 3099264, No. 3104409, and No. 3104434; and U.S. Pat. No. 7,201,945 and No. 7,320,713) to produce clothing. According to these patents and products introduced by Phild Co. Ltd., the purpose of developing these healthy fiber products is only to help to relieve fatigue, muscle pain, increase blood circulation and cleansing, and provide muscle relaxation.

In a similar fashion, various other publications and technologies describe many health-enhancing applications using Titanium metal and Titanium oxide. Examples include using the optical catalysis characteristics of Titanium metal and its oxide to produce far-infrared rays, or using Titanium oxide in deodorizing and antibacterial applications, or the like. However, to date, none of these publications and patent technologies involves the application of Titanium metal fine-particles to create a Titanium metal fine-particles layer on, or close to, human body skin for increasing the effect of the germicidal medicines which are used for human skin dermatosis, skin infection and traumatism.

### SUMMARY OF THE INVENTION

The purpose of the present invention is therefore to provide an application of Titanium metal fine-particles in a manner so as to increase the effect of the germicidal medicines which are used for human skin dermatosis, skin infection and traumatism. Based on the application realities and appearances, it can be speculated that the Titanium pure metal fine-particles produce a regulating effect on the micro bio-current and micro bio-magnetic field in/on the horny layer of skin (stratum corneum), capillary structure under skin and the musculature structure under skin, when these fine-particles, which are of almost the same electrical polarity and scattered evenly in a certain area, form a layer of particles on or close to body skin. It is imaginable the micro bio-current and micro bio-magnetic fields which are disordered due to damage of the skin caused by the dermatophyte and the inflammation on traumatism separately in or on the horny layer of skin, capillary under skin and musculature under skin, will be regulated by the Titanium pure metal fine-particles layer. This regulating effect will be in opposition to the disordered micro bio-current and micro bio-magnetic fields caused by the dermatophyte and the inflammation on traumatism, and it will effectively restrain the multiplication and breeding of bacteria in/on the skin infection and/or traumatism. If this regulating effect is applied for a sufficient length of time, it will greatly accelerate skin and dermic-organism metabolism and promote capillary purification. Meanwhile it will also establish an environment to sufficiently restrain the multiplication and breeding of bacteria in/on skin infections and/or traumatism. In this environment, adding the effect of the germicidal medicines which are used for human skin dermatosis, skin infection and traumatism, the multiplication and breeding of bacteria will be relatively quickly restrained. Furthermore, because of the acceleration of skin and dermic-organism metabolism and the promotion of capillary purification, compared with only the application of the germicidal medicine, the recovery and subsequent curing of the skin wound and traumatism will be completed within a shorter length of time. Finally, these effects will be demonstrated by the increased effect of the germicidal medicines when used for human skin dermatosis, skin infection and traumatism.

This invention therefore provides the use of Titanium metal fine-particles for increasing the effect of the germicidal medicines used for human skin dermatosis, skin infection and traumatism.

As such, the invention provides the use of Titanium metal fine-particles for increasing the effect of the germicidal medicines used for human skin dermatosis, skin infection and traumatism. Preferred germicidal medicines are the medicines used for antifungal, treating dermatophyte and anti-inflammation on skin-infection or traumatism. The dermatophyte, hereby, indicates specifically some funguses, anaerobe, rosary fungus, Gardner fungus, etc. The inflammation on traumatism, hereby, indicates that which is caused mainly by Gram positive bacteria (such as staphylococcus aureus rosenbach, streptococcus, hemolytic streptococcus, enterococcus), and also by some Gram negative bacteria (such as Escherichia coli, pseudomonas aeruginosa, or proteus species).

Further, the invention provides the use of Titanium metal fine-particles for increasing the effect of the germicidal medicines used for human skin dermatosis, skin infection and traumatism, wherein the Titanium metal fine-particles is pure Titanium metal particles with an average diameter size on the order of 10̂-5˜10̂-9 meter.

Still further, the invention provides the use of Titanium metal fine-particles for increasing the effect of the germicidal medicines used for human skin dermatosis, skin infection and traumatism, wherein the Titanium metal fine-particles are to be bonded with, or to be contained in, the fibrous or non-fibrous substrate which is used as body-dressing or skin-cover sheet. As the Titanium metal fine-particles are scattered evenly in the fibrous or non-fibrous substrate, the Titanium metal fine-particles will form a layer of particles on, or close to, the body's skin when wearing the body-dressing or applying the skin-cover sheet to the human body. This Titanium metal fine-particles layer will increase the effect of the germicidal medicines used for human skin dermatosis, skin infection and traumatism.

Also, the present invention provides the use of Titanium metal fine-particles for increasing the effect of the germicidal medicines used for human skin dermatosis, skin infection and traumatism, wherein the fibrous substrate is a woven or non-woven fabric substrate; or wherein the non-fibrous substrate is made from nylon, plastic or other softened sheet material.

The present invention also provides the use of Titanium metal fine-particles for increasing the effect of the germicidal medicines used for human skin dermatosis, skin infection and traumatism, wherein the application of woven or non-woven fibrous substrate is through the use of a sock, a glove, finger/toe-sleeves, or the like, that contains or is bonded with, the Titanium metal fine-particles, together with the general antifungal medicines used for dermatosis and athlete's foot (for example), so as to increase the effect of the medicines, wherein the fibrous sock, glove, and finger/toe-sleeves are used regardless of whether the socks, gloves or finger/toe-sleeves are finger- or toe-separating or non-separating type. Also, the invention contemplates the use of woven or non-woven fibrous underwear/underclothing, shirts, fitness wear, scarf, masks and ear-covers, or the like, that contains, or is bonded with, the Titanium metal fine-particles, together with the general antifungal medicines used for dermatosis, or together with general anti-inflammation/antibiotics medicines used for skin wound and traumatism, or together with some skin care medicines so as to increase the effect of the medicine.

Further, the invention also provides the use of Titanium metal fine-particles for increasing the effect of the germicidal medicines used for human skin dermatosis, skin infection and traumatism, wherein the Titanium metal fine-particles is provided as a Titanium metal micro-dispersion in water or volatile liquid in which the Titanium metal fine-particles has been dispersed. Preferably, the Titanium metal fine-particles are preferably the Titanium metal powder which comes from the Titanium metal micro-dispersion in water or other volatile liquid, and resulting from a process of concentration, distillation, filtration or volatilization.

The present invention also provides for the use of Titanium metal fine-particles for increasing the effect of the germicidal medicines used for human skin dermatosis, skin infection and traumatism, wherein the application of woven or non-woven fibrous skin-cover sheet which have been treated with the Titanium metal micro-dispersion water or volatile liquid, will be used for medical covering materials or bandages on skin wounds and traumatism, wherein the skin-covering sheet includes, but is not limited to, medical gauze, medical bandage, bandage supporter, etc. Preferably, the treatment comprises using the Titanium metal micro-dispersion in water, volatile liquid or process solvent containing the Titanium metal micro-dispersion water to imbue, spray, shower and paint with, or on, the skin-cover sheet, and then remove the moisture or solvent in order to leave dispersed Titanium fine-particles within the fibrous structure of skin-cover sheet and/or on its surface.

The present invention also provides for the use of Titanium metal fine-particles for increasing the effect of the germicidal medicines used for human skin dermatosis, skin infection and traumatism, wherein a Titanium metal micro-dispersion in water is used as the carrier or substrate for the germicidal medicine, or the application of the Titanium metal powder which comes from the Titanium metal micro-dispersion in water or volatile liquid after the process of concentration, distillation, filtration or volatilization, and used as a part of the carrier or substrate of germicidal medicine, will produce the antifungal or inflicting dermatophyte medicine used for dermatosis, athlete's foot and some skin infections. As described above, the medicine will preferably be in the style of a liquid, half-liquid or cream, powder or spray powder, as per the style of medicine carrier or substrate.

The present invention also provides for the use of Titanium metal fine-particles for increasing the effect of the germicidal medicines used for human skin dermatosis, skin infection and traumatism, wherein the Titanium metal micro-dispersion water which is used as the carrier or substrate of germicidal medicine, or the application of the Titanium metal powder which comes from the Titanium metal micro-dispersion water after the process of concentration, distillation, filtration or volatilization, and is used as a partial of the carrier or substrate of germicidal medicine, will produce the medicine used for anti-inflammation/antibiotics on skin wound and traumatism. As described above, the medicine will preferably be in style of a liquid, half-liquid or cream, powder or spray powder as per the style of medicine carrier or substrate.

Therefore, the key point of the present invention is to create a Titanium metal fine-particles layer on, or close to, the body skin, and to keep this layer present for a certain time, so as to provide the necessary basic condition for increasing the effect of germicidal medicines. Following this basic condition only, the Titanium metal fine-particles layer will typically increase the effect of medicines greatly (e.g. from 50% to over 100%) when compared with applying the medicine only. By using the fibrous or non-fibrous body-dressing and skin-cover sheet as described in above, or by using the medicines in the styles of liquid, cream, powder or spray powder with the Titanium metal fine-particles in their carrier or substrate as described hereinabove, the Titanium metal fine-particles dispersed evenly within them will form a particles layer on or close to body and skin, and will keep the layer for the specified time for the body-dressing or skin-cover sheet, or the liquid, cream, powder and spray powder medicines, when applied over the human body, or when applying these medicines on human skin.

### DETAILED DESCRIPTION OF THE INVENTION

The invention will now be described below with regards to the details of the application methods. The skilled artisan will understand that these application methods are used for explaining the content of this invention only and not for limiting the scope of the present invention. It is also to be understood that, after reading the content of this application, that any equivalent type/style of applying methods will still fall within the scope described in the claims of the present invention, even through some modifications or changes have been added to this invention.

This invention provides an application of the Titanium metal fine-particles for increasing the effect of the germicidal medicines which are used for human skin dermatosis, skin infection and traumatism.

Preferably, the germicidal medicines are the medicines used for antifungal, inflicting dermatophyte and anti-inflammation on skin-infection or traumatism. The dermatophyte, hereby preferably indicates specifically some funguses, anaerobe, rosary fungus, Gardner fungus, etc.; and the inflammation on traumatism, hereby, indicates that is caused mainly by Gram positive bacteria (such as: staphylococcus aureus rosenbach, streptococcus, hemolytic streptococcus, enterococcus), and also by some Gram negative bacteria (such as: Escherichia coli, pseudomonas aeruginosa, proteus species).

The preferred Titanium metal fine-particles are pure Titanium metal particles with an average diameter size on the order of 10̂-5˜10̂-9 meter. Titanium metal fine-particle are preferably bonded with, or contained in, a fibrous or non-fibrous substrate which is used as body-dressing or skin-cover sheet. As the Titanium metal fine-particles are scattered evenly in the fibrous or non-fibrous substrate, the Titanium metal fine-particles will form a particles layer on, or close to, body skin when wearing the body-dressing or applying the skin-cover sheet to human body. This Titanium metal fine-particles layer increases the effect of germicidal medicines used for human skin, skin infection and traumatism.

The fibrous substrate is woven or non-woven fabric substrate; wherein the non-fibrous substrate is made from nylon, plastic or other softened sheet material.

The Titanium metal fine-particles are used for increasing the effect of the germicidal medicines used for human skin dermatosis, skin infection and traumatism as described above, wherein the application of woven or non-woven fibrous sock, glove and finger/toe-sleeve, which contain or which are bonded with the Titanium metal fine-particles, together with general antifungal medicines used for dermatosis and athlete's foot will increase the effect of medicines, wherein the fibrous sock, glove and finger/toe-sleeve are finger/toe are separating or non-separating type. The application of woven or non-woven fibrous underwear/underclothes, shirt, fitness wear, scarf, mask and ear-cover, which contains or is bonded with the Titanium metal fine-particles, together with general antifungal medicines used for dermatosis, or together with general anti-inflammation/antibiotics medicines used for skin wound and traumatism, or together with some skin care medicines, will increase the effect of the medicines.

The application of the Titanium metal fine-particles is used for increasing the effect of the germicidal medicines which are used for human skin dermatosis, skin infection and traumatism as described above, wherein the Titanium metal fine-particles are the Titanium metal micro-dispersion water or volatile liquid in which the pure Titanium metal fine-particles are dispersed and with an average dimension size on the order of 10̂-5˜10̂-9 meter; wherein the Titanium metal fine-particles are the Titanium metal powder which comes from the Titanium metal micro-dispersion water or volatile liquid and treated by the process of concentration, distillation, filtration or volatilization.

The application of the Titanium metal fine-particles is used for increasing the effect of the germicidal medicines which are used for human skin dermatosis, skin infection and traumatism as described above, the application of woven or non-woven fibrous skin-cover sheet, which is treated with the Titanium metal micro-dispersion water or volatile liquid, will be for medical cover material and bandage on skin wound and traumatism, wherein the skin-cover sheet includes and is not limited to medical gauze, medical bandage, bandage supporter, etc.; wherein, the treatment indicates using the Titanium metal micro-dispersion water, volatile liquid or process solvent contained with the Titanium metal micro-dispersion water to imbue, spray, shower and paint with/on the skin-cover sheet, then by removing the moisture or solvent to leave dispersed Titanium fine-particles within the fibrous structure of skin-cover sheet and on its surface.

The application of the Titanium metal fine-particles is used for increasing the effect of the germicidal medicines which are used for human skin dermatosis, skin infection and traumatism as described above, the application of the Titanium metal micro-dispersion water which is used as the carrier or substrate of germicidal medicine, or the application of the Titanium metal powder which comes from the Titanium metal micro-dispersion water or volatile liquid after the process of concentration, distillation, filtration or volatilization, and used as a partial of the carrier or substrate of germicidal medicine, will produce the antifungal or inflicting dermatophyte medicine used for dermatosis, athlete's foot and some skin infections. As described above, the medicine will preferably be in style of liquid, half-liquid or cream, powder or spray powder as per the style of medicine carrier or substrate.

The application of the Titanium metal fine-particles is used for increasing the effect of the germicidal medicines which are used for human skin dermatosis, skin infection and traumatism as described above, the application of the Titanium metal micro-dispersion water which is used as the carrier or substrate of germicidal medicine, or the application of the Titanium metal powder which comes from the Titanium metal micro-dispersion water after the process of concentration, distillation, filtration or volatilization, and is used as a partial of the carrier or substrate of germicidal medicine, will produce the medicine used for anti-inflammation/antibiotics on skin wound and traumatism. As described in above, the medicine will preferably be in style of liquid, half-liquid or cream, powder or spray powder as per the style of medicine carrier or substrate.

### Examples

### Application Method 1:

Using a body-dressing or skin-cover sheet, in which the Titanium fine-particles are contained or bonded with, and the body-dressing or skin-cover sheet could be fibrous (woven or non woven) or non-fibrous substrate, to cover body or skin area which is necessary after applying germicidal medicine, so as to create a Titanium fine-particles layer covering on this body or skin area. The layer covering could be attached to the skin, but also could be a close to skin (likely to be separated by medicine layer or other thinner medical isolation material), and it kept attached or close to skin for a certain time (at least one hour per day).

The application process flow is as shown below:

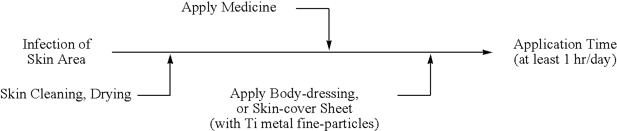

The longer the time the dressing or skin-cover sheet is kept attached or close to the skin, the larger the increase in the medical effects.

Application details: Based on the directions of the medicine itself, or on the instruction of doctor, apply the medicine to the body. Then, use the body-dressing or skin-cover sheet, in which the Titanium fine-particles are contained in or bonded with, and replace the body-dressing or skin-cover sheet according to the specified frequencies for change of the medicine. Following this procedure, the recovery and the cure of the skin wound and/or traumatism will be completed within a shorter time.

It is noted that considering the general necessary of providing fresh air and oxygen for skin and dermic-organism metabolism, a body-dressing or skin-cover sheet made of a fibrous substrate which contains and/or is bonded with the Titanium fine-particles, is preferred.

Applying case 1: Using fibrous toe-separating socks which are made by Phild Co. Ltd., specifically for helping to relieve fatigue and pain in a foot muscle, apply the sock together with the medicine prescribed by a doctor for the treatment of Athlete's foot.

Detail applying method: Before sleeping every day, apply the prescribed medicine according to doctor and/or the medicine instruction, and then wear the fibrous toe-separating socks while sleeping. In the morning, wake-up and remove the toe-separating and change to general socks. By using this method every day, even a patient who has serious symptoms will begin to feel the effect of treatment after 15 days. Within one to two months the patient will be 90% recovered, and over longer time periods, the patient will be recover completely. If continuously used in this manner, as described above, after recovery, the treatment will be helpful for preventing a repeat of the condition. (5 person comparison test)

Applying case 2: Using traumatism medical bandage which is made from a fibrous inside material, and which contains and/or is bonded with the Titanium fine-particles, after disinfecting in high temperature, apply the bandage together with anti-inflammation medicine for covering and bandaging of skin wound and traumatism.

Detail applying method: Take traumatism area medical disinfectant medicine, and apply anti-inflammation medicine. Cover with 1 to 3 layers of cotton medical gauze for absorbing the bloody and body liquid coming from the skin wound, then bandage the skin wound with a skin bandage as described in above. Comparing the same traumatism and same medicine treatment, using a fibrous bandage containing the Titanium fine-particles will make the traumatism recover faster by about 1 to 3 days, depending on the traumatism situation. (2 person comparing test)

### Application Method 2:

Using the Titanium metal micro-dispersion water or volatile liquid, or the Titanium metal fine powder from the Titanium metal micro-dispersion water or volatile liquid as the carrier or substrate of germicidal medicine, mix the particles with the components of the medicine to produce a new type of medicine which is enriched with Titanium metal fine-particles. When applying these new type of medicines to infected areas, a Titanium fine-particles layer is created on the skin surface together, with the medicine components. In use, the user applies the enriched medicine to the infected area for a certain time (preferably at least 1 hour per day). When compared with same medicine without the Titanium metal fine-particles in the carrier or in the medicine substrate, the modified medicine has a better effect.

Applying idea 1: Using the Titanium metal micro-dispersion in water or volatile liquid and the Titanium metal fine powder to produce new type medicines for Athlete's foot and other dermatosis.

Applying idea 2: Using the Titanium metal micro-dispersion water or volatile liquid and the Titanium metal fine powder to produce new types of medicines for anti-inflammation of skin wounds and traumatism.

As explained in detail and described by examples above, using applying idea 1, compared with existing medicines treatment only, the treatment of medicine (same ingredients and dosage) together with the Titanium metal fine-particles layer increases the healing effect over 50% to 200˜300%. The evaluation of the effect is studied by comparing the days of achieving the same recovery level for the same medicine dosage.

Using apply idea 2, compared with existing medicines, the new type medicine (same medicine ingredients and dosage) will show more improved effects of over 50% to 200˜300%, even when the same level of medicine components is used, because of the use of the Titanium metal fine-particles layer. The evaluation of the effect is studied by comparing the days of achieving the same recovery level for the same medicine dosage.

Case Study 1: Over two patients used an existing Athlete's foot medicine for treatment. They applied the medicine once per day as doctor's instruction. Although the medicine showed some effects in 7 to 10 days, their Athlete's foot was still not recovered completely, even after 1 to 2 months. Later, they stopped the medicine and their condition eventually went back to its original condition. In contrast, when they used the fibrous toe-separating socks that contained the Titanium metal fine-particles, together with applying the same Athlete's foot medicines in same way (applying once per day), the treatment showed obvious effects from the 7th to 10th days. After 20 days, their Athlete's foot symptoms disappeared completely. They continuously used the special fibrous toe-separating socks, together with the Athlete's foot medicine, until the medicine was completely used. To date, no recurrence of the condition has been observed.

Case Study 2: Two persons do a compare test for the same skin wound and traumatism. One person takes a traumatism area medical disinfectant and applies an anti-inflammation medicine, and then bandages the wound with prior art general bandaging material on the top of cotton medical gauze. The second person takes traumatism area medical disinfectant and applies the same anti-inflammation medicine, and then bandages the wound with the new bandaging material, which includes fibers containing the Titanium fine-particles, on top of cotton medical gauze. For the same skin traumatism and the same anti-inflammation medicine, the person who uses the new bandaging material which uses fibrous materials containing the Titanium fine-particles, has recovered and closed his skin wounds in only 2.5 days and 4 days respectively. In contrast, another person, who used existing prior art bandaging material, needs 4 days and 7 days respectively for recovering and closing his skin wounds to same condition.

1. Use of Titanium metal fine-particles for increasing of effect of the germicidal medicines used for human skin dermatosis, skin infection and traumatism.

2. The use as claimed in claim 1, wherein the germicidal medicines are the medicines used for antifungal, inflicting dermatophyte and anti-inflammation on skin-infection or traumatism; wherein the dermatophyte, hereby, indicates some funguses, anaerobe, rosary fungus, Gardner fungus, etc.; and the inflammation on traumatism is caused mainly by Gram positive bacteria (such as staphylococcus aureus rosenbach, streptococcus, hemolytic streptococcus, enterococcus), and also by some Gram negative bacteria (such as Escherichia coli, pseudomonas aeruginosa, proteus species).

3. The use as claimed in claim 1, wherein the Titanium metal fine-particles is pure Titanium metal particles with an average diameter size on the order of 10̂-5˜10̂-9 meter.

4. The use as claimed in claim 1, wherein the Titanium metal fine-particles are to be bonded with or to be contained in the fibrous or non-fibrous substrate which is used as body-dressing or skin-cover sheet; ;and the Titanium metal fine-particles are scattered evenly in the fibrous or non-fibrous substrate, so that the Titanium metal fine-particles will form a particles layer on or close to the skin when wearing the body-dressing or applying the skin-cover sheet to human body.

5. The use as claimed in claim 4, wherein the fibrous substrate is woven or non-woven fabric substrate;

6. The use as claimed in claim 5 wherein the non-fibrous substrate is made from nylon, plastic or other soften sheet material.

7. The use as claimed in claim 4, comprising applying a woven or non-woven fibrous sock, glove and finger/toe-sleeve, that contains, or is bonded with, Titanium metal fine-particles, together with general antifungal medicines used for dermatosis and athlete's foot in order to increase the effect of the medicines, wherein the fibrous sock, glove and finger/toe-sleeve are finger/toe separating or non-separating type.

8. The use as claimed in claim 4, comprising applying a woven or non-woven fibrous underwear/underclothes, shirt, fitness wear, scarf, mask and ear-cover, that contains, or is bonded with, the Titanium metal fine-particles, together with general antifungal medicines used for dermatosis, or together with general anti-inflammation/antibiotics medicines used for skin wound and traumatism, or together with some skin care medicines to increase the effect of the medicines.

9. The use as claimed in claim 1, wherein the Titanium metal fine-particles are the Titanium metal micro-dispersion in water or a volatile liquid in which the Titanium metal fine-particles are dispersed; and wherein the Titanium metal fine-particles are the Titanium metal powder which comes from the Titanium metal micro-dispersion water or volatile liquid and treated by the process of concentration, distillation, filtration or volatilization.

10. The use as claimed in claim 9, comprising applying woven or non-woven fibrous skin-cover sheet, which is treated with the Titanium metal micro-dispersion water or volatile liquid, for medical cover material and bandage on skin wound and traumatism; wherein, the skin-cover sheet includes and is not limited to medical gauze, medical bandage, bandage supporter, etc.; and wherein, the treatment indicates using the Titanium metal micro-dispersion water, volatile liquid or process solvent contained with the Titanium metal micro-dispersion water to imbue, spray, shower and paint with/on the skin-cover sheet, then by removing the moisture or solvent to leave dispersed Titanium fine-particles within the fibrous structure of skin-cover sheet and on its surface.

11. The use as claimed in claim 7, comprising applying the Titanium metal micro-dispersion water which is used as the carrier or substrate of germicidal medicine, or applying the Titanium metal powder which comes from the Titanium metal micro-dispersion water or volatile liquid after the process of concentration, distillation, filtration or volatilization, and is used as a partial of the carrier or substrate of germicidal medicine, to produce the antifungal or inflicting dermatophyte medicine used for dermatosis, athlete's foot and some skin infections.

12. The use as claimed in claim 11 wherein the medicine will be in style of liquid, half-liquid, cream, powder or spray powder as per the style of medicine carrier or substrate.

13. The use as claimed in claim 9, comprising applying the Titanium metal micro-dispersion water which is used as the carrier or substrate of germicidal medicine, or applying the Titanium metal powder which comes from the Titanium metal micro-dispersion water after the process of concentration, distillation, filtration or volatilization, and which is used as a partial of the carrier or substrate of germicidal medicine, to produce the medicine used for anti-inflammation/antibiotics on skin wound and traumatism.

14. The use as claimed in claim 13 wherein the medicine will be in style of liquid, half-liquid or cream, powder or spray powder as per the style of medicine carrier or substrate.

15. A germicidal medicine used for treatment of human skin dermatosis, skin infection or traumatism, wherein said medicine comprises Titanium metal fine-particles for increasing of effect of the germicidal medicine.
